(19) United States
(12) Patent Application Publication
(10) Pub. No.: US 2019/0350930 A1
(43) Pub. Date: Nov. 21, 2019

(54) COMPOSITION FOR CONTROLLING GASTROESOPHAGEAL REFLUX DISEASES

(71) Applicant: bitop AG, Witten (DE)

(72) Inventor: Andreas Bilstein, Bergheim (DE)

(73) Assignee: bitop AG, Witten (DE)

(21) Appl. No.: 16/312,805

(22) PCT Filed: Jun. 27, 2017
(86) PCT No.: PCT/EP2017/065799

§ 371 Date: Dec. 21, 2018

(30) Foreign Application Priority Data
Jun. 29, 2016 (DE) 10 2016 111 882.6

(51) Int. Cl. A61K 31/505; A61P 1/04

(52) U.S. Cl. CPC A61K 31/505; A61P 1/04

(57) ABSTRACT

The invention relates to a composition containing as active agent ectoine, hydroxyectoine and/or salts, esters or amides of these compounds for use in a method for the treatment and/or prophylaxis of gastroesophageal reflux diseases, inflammation of and damage to the gastric or duodenal mucosa and/or of gastric or duodenal ulcers.

The invention relates to a composition for the treatment and prophylaxis of gastroesophageal reflux diseases, inflammation of and damage to the gastric or duodenal mucosa and/or gastric or duodenal ulcers.

Gastroesophageal reflux diseases (GERD), also known as heartburn (pyrosis), are a common phenomenon. In western industrialized countries, the problem occurs at least once a week among adults in about 10 to 20% of the population. In East Asia the prevalence ranges between 2.5 and 7.8%, and in the USA 20% of the adult population is affected weekly, 7% even daily. The disease is primarily due to the fact that gastric acid from the stomach enters the esophagus. Aside from gastric acid, the ingress of other contents of the stomach into the esophagus also plays a role, for example the digestive enzyme pepsin, a peptidase that serves to digest proteins in food. The damaging effect of gastric acid is intensified by pepsin.

For those affected, the disease is a great burden, on the one hand because it causes a sharp burning sensation in the esophagus, and on the other hand because it is unpleasant in daily contact with people, since the acid eructation can hardly be controlled. More often than not, patients feel a burning sensation that even reaches up to the throat. The common treatment option usually adopted is the use of proton pump inhibitors (PPIs) such as omeprazole and histamine H2 receptor antagonists (H2Ra). Both substance classes are intended to suppress the production of gastric acid. Another treatment option is the administration of antacids, i.e. gastric acid neutralizing substances. Alginates provide for the formation of a viscous foam in the stomach, which prevents the reflux of gastric acid into the esophagus.

Gastroesophageal reflux diseases are often associated with cardia insufficiency. This is a malfunction of the sphincter muscle (esophagus sphincter) that separates the esophagus from the stomach causing contents of the stomach to flow back into the esophagus. Other causes may include an excessive gastric acid production or a defective peristalsis of the esophagus. The problem occurs particularly frequently at night, i.e. when affected persons are in a lying position. Also sweet desserts or the consumption of tobacco and alcohol can promote the occurrence of reflux symptoms.

Gastroesophageal reflux diseases can manifest themselves as non-erosive reflux diseases (NERD), in which no damage to the mucous membrane of the esophagus is found, but also as erosive reflux diseases (reflux esophagitis; erosive esophagitis (EE); erosive reflux disease (ERD)). In the latter, the mucous membrane in the esophagus changes demonstrably and mucous membrane damage can be detected. Bleeding and ulcers may occur in the area of transition between stomach and esophagus. Another complication of gastroesophageal reflux diseases can be a narrowing of the esophagus, which in turn leads to difficulties in swallowing.

A further stage may be a Barrett's esophagus (endobrachyesophagus), in which a metaplastic transformation of the epithelium of the esophagus can be observed, with a multi-layered squamous epithelium of the esophagus transforming into a single-layered prismatic cylindrical epithelium in the distal region. This transformation can be completely circular, especially in the area of the gastroesophageal transition, i.e. the transition from the stomach to the esophagus. Although cylinder epithelium is more resistant to gastric acid and the gastric enzyme pepsin, there is nevertheless a risk of dysplasia. A Barrett's esophagus may therefore be a preliminary stage in the development of esophageal carcinoma (Barrett's carcinoma) and must therefore be scrutinized and monitored. In addition, a Barrett's esophagus may lead to the formation of ulcers.

As mentioned above, various treatment options are available for gastroesophageal reflux diseases. Even with the most effective method of treatment currently available, the administration of proton pump inhibitors, the symptoms of the disease, which are unpleasant for the patient, are not always completely suppressed. Aside from this, there may be side effects such as osteoporosis due to mineral deficiency or intestinal inflammation, vitamin B12 deficiency and negative effects on the liver and kidneys.

It is, therefore, the objective of the invention to provide alternative substances or compositions that are suitable for the treatment and prophylaxis of gastroesophageal reflux diseases and pyrosis.

Surprisingly, it has been discovered that ectoine, hydroxyectoine and salts, esters and amides of ectoine/hydroxyectoine are capable of bringing about significant improvements in gastroesophageal reflux diseases and pyrosis. It could be demonstrated that ectoine is able to remedy the negative effects of acid and pepsin on squamous epithelial cells.

It has also been shown that ectoine, hydroxyectoine as well as salts, esters and amides of ectoine/hydroxyectoine can guard against and provide therapy with respect to damage of the gastric or intestinal epithelium. This applies in particular to the treatment and prophylaxis of gastritis. The aggressive gastric acid can attack the stomach mucosa, for example if the production of the protective mucus layer is disturbed by external factors. Surprisingly, it has turned out that ectoine and its derivatives are capable of preventing or treating gastritis. Gastritis can develop as a result of reflux esophagitis.

Gastritis can lead to the formation of stomach ulcers (ulcus ventriculi), which, ultimately, are as well due to the aggressive gastric acid in the event the stomach wall and the gastric mucosa are not sufficiently protected against gastric acid. One of the damaging factors, for example, is an overproduction of gastric acid. As a rule, the formation of gastric ulcers is, among other things, attributable to damage to the mucous membrane of the stomach; in this respect, the protection of the mucous membrane of the stomach also provides protection against gastric ulcers.

Damage to the epithelium of the duodenum can also be prevented by ectoine, hydroxyectoine or corresponding salts, esters or amides, which is why the substances are well-suited for the prophylaxis as well as treatment of inflammations of the mucous membrane of the duodenum. Similar to the gastric mucosa, this is a single-layer cylindrical epithelium. The duodenum is the first part of the small intestine that adjoins the stomach. Since it is exposed to the highly caustic stomach contents, which are mixed with digestive enzymes such as pepsin, inflammation of and damage to the duodenal mucosa can occur. Moreover, bile from the liver and gall bladder as well as pancreatic enzymes are supplied to the food in the duodenum. Damage to the duodenal mucosa can lead to a duodenal ulcer (ulcus duodeni), which affects approx. 2 to 10% of people in the course of their lives. The development of a duodenal ulcer is also based on an imbalance between substances attacking the mucous membrane such as gastric acid and certain proteases as well as factors protecting the mucous membrane such as an adequate formation of mucus. The protection of the mucous membrane of the duodenum is therefore of significance for the prophylaxis and treatment of duodenal ulcers. It has been found that ectoine, hydroxyectoine and their derivatives referred to hereinbefore are effectively put to use for prophylactical purposes to prevent the development of both gastric and duodenal ulcers and are also effectively used in the treatment of gastric/duodenal ulcers. Even if the damage to the mucous membrane of the stomach/duodenum is less advanced and has not yet resulted in the development of an ulcer, any damage (erosion) occurring in that area can be treated and prevented.

Ectoine and hydroxyectoine are tetrahydropyrimidine derivatives which are synthetized under stress conditions in extremophilic, especially halophilic microorganisms. Various applications or uses have been described hitherto for ectoine and hydroxyectoine, for example as moisturizers, for the treatment of the vascular leak syndrome (VLS) (DE 10 2006 056 766 A1) or for the treatment of neurodermatitis (DE 103 30 243 A1).

The systematic name of ectoine is 2-methyl-1,4,5,6-tetrahydropyrimidine-4-carboxylic acid and of hydroxyectoine 5-hydroxy-2-methyl-1,4,5,6-tetrahydropyrimidine-4-carboxylic acid.

The structure of natural L-ectoine ((S)-2-methyl-1,4,5,6-tetrahydropyrimidine-4-carboxylic acid) is illustrated below:

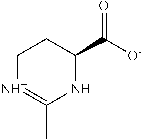

The structure of natural hydroxyectoine ((4S,5S)-5-hydroxy-2-methyl-1,4,5,6-tetrahydropyrimidine-4-carboxylic acid) is indicated hereunder:

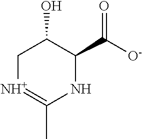

The use of the stereoisomers indicated is preferred but not obligatory, i.e. other stereoisomers or racemates may also be employed.

The composition proposed by the present invention can be used for the treatment and/or prophylaxis of gastroesophageal reflux diseases or pyrosis of varying severity, i.e. both for non-erosive reflux diseases as well as for reflux esophagitis in which damage to the mucous membranes of the esophagus has already been detected. Treatment options include Barrett's esophagus, which is a serious disease associated with an increased risk of cancer.

Typically, the composition is an aqueous solution, in most cases intended for oral administration.

Pharmacologically compatible salts of the ectoine/hydroxyectoine embrace alkaline or alkaline-earth salts, in particular the salts of potassium, sodium, magnesium and calcium but also salts with organic bases such as, for example, with non-toxic aliphatic or aromatic amines.

Through the reaction of the carboxyl group of the ectoine/hydroxyectoine with alcohols or amines relevant esters or amides can be obtained which may also be employed within the scope of the invention. In this case, the COOH group of ectoine/hydroxyectoine is replaced by a carboxylic acid ester function COOR or a carboxylic acid amide function CONHR′ or CONR′R″, where R, R′, R″ independently of one another represent saturated or unsaturated, straight-chain or branched alkyl, cycloalkyl, aryl, heteroaryl, alkylaryl, arylalkyl, alkoxyalkyl, alkylthioalkyl, aryloxyalkyl or arylthioalkyl groups. In particular, it may be a C₁, C₂, C₃, C₄, C₅, C₆, C₇, C₈, C₉, C₁₀, C₁₁, C₁₂, C₁₃, C₁₄, C₁₅, C₁₆, C₁₇ or C₁₈ alkyl group. In case of hydroxyectoine also the hydroxy group may be subjected to a reaction with a carboxylic acid of varying chain length to form a relevant ester. The respective esters or amides may also be present in ionic or zwitterionic form, that is, the invention embraces the use of salts of said esters or amides.

Likewise, the ectoine amide of 2-hydroxy-5-aminobenzoic acid may be used as well. The structural formula is as follows:

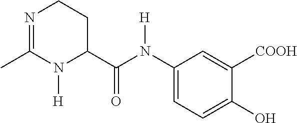 [SMILES: CC1=NCCC(C(=O)Nc2ccc(O)c(C(=O)O)c2)N1]

Therefore, this is the 2-methyl-1,4,5,6-tetrahydropyrimidine-4-carboxylic acid amide of the 2-hydroxy-5-aminobenzoic acid. Preferably, it is the relevant amide of the L-ectoine: (S)-2-methyl-1,4,5,6-tetrahydropyrimidine-4-carboxylic acid amide. The use of hydroxyectoine amide of the 2-hydroxy-5-aminobenzoic acid is also conceivable.

The composition may contain customary auxiliary substances, for instance carrier agents, preservation agents, bactericides, solutizers, vitamins, stabilizers, anti-foaming substances, osmotically active substances, colorants, surfactants, emulsifiers, moisturizing substances, viscosity increasing agents or the like. In the event of an administration in solid form, for example by means of powder inhalers, it is recommendable that only carrier substances are used that are easily resorbed and non-irritating such as micronized lactose.

For example, preservation agents include thiomersal, organic mercury compounds such as phenylmercury, benzalkonium chloride, chlorhexidine, benzyl alcohol, glucose, ethanol and quaternary ammonium salts.

The formulations within the scope of the invention may also contain suitable buffer systems or other auxiliary substances for pH adjustment in order to reach and maintain a desired pH value. Typically, the pH values range between 4 and 8, preferably between 5 and 7.5, more preferred is a value of approx. 7. Suitable buffer systems are citrate, phosphate, TRIS, glycine, borate, acetate. These buffer systems may be produced from substances such as citric acid, monosodium phosphate, disodium phosphate, glycine, boric acid, sodium tetraborate, acetic acid or sodium acetate.

While the compositions may contain further active agents it is, however, easily possible and sufficient for the treatment or prevention of the ageing symptoms to use compositions that only contain as active substances ectoine, and/or hydroxyectoine, respectively salts, esters or amides thereof.

The concentration of ectoine/hydroxyectoine and/or corresponding salts, esters or amides can, in particular, be in a range of between 10 and 500 mM, preferably between 50 and 500 mM, especially preferred between 100 and 500 mM and/or between 100 and 200 mM. These concentrations have proven to be suitable for achieving the inventive effect. In particular, the proportion of ectoine/hydroxyectoine and/or corresponding salts, esters or amides in the composition may be in a range from 0.05 to 20% w/w, preferably from 0.1 to 10% w/w. A good effect could be observed, for example, in a range between 0.5 and 2% w/w.

The composition may be provided in the form of a solution, preferably an aqueous solution. However, it may as well be provided in the form of a suspension, emulsion or microemulsion.

In order to improve the application and the shelf life of the composition provided in accordance with the invention, the composition containing the active ingredient may also be administered encapsulated in nanostructures or in the form of liposomes. This is particularly advantageous if the composition does not contain any preservative agents. Suitable methods for the encapsulation and production of liposomes are generally known from the state of the art.

### EXAMPLE

With a view to demonstrating the effect of ectoine to counteract pyrosis and other damage to the epithelium in the gastrointestinal tract, an in vitro cell culture model with the mucus-forming epithelial cells TR146 was used to simulate the situation in the esophagus. The cells were treated with acid solutions containing the digestive enzyme pepsin and following this treated with ectoine.

TR146 cells were added to a microplate having 96 wells, with the cells being allowed to grow until the surface was almost completely covered. The cells were then treated for 10 min. with a diluted acidic HCl solution together with pepsin in a cell culture medium. The concentration of pepsin was 2 U/ml. Following this, the cells were washed with PBS (phosphate buffered saline) and incubated overnight either with ectoine or for control purposes with pure solvent. After incubation, a cell count determination was carried out using a neutral red cytotoxicity assay for which a cyanine-based nucleic acid dye was employed. The assay is based on the fact that the plasma membrane of damaged or dead cells becomes permeable to the dye and the dye binds to the DNA of the cell, which is associated with a corresponding increase in fluorescence.

For negative control, cells were treated with 0.1% sodium dodecyl sulfate (SDS) solution as denaturing agent. Other cells (full medium) were not treated with acid/pepsin. The results are shown in FIG. 1. It is evident from the FIGURE that 50 mM of ectoine had an excellent effect with respect to the regeneration of the cells. The cell viability of cells treated with ectoine was comparable to that of cells that were not subjected to a treatment with an acidic pepsin solution.

1. Composition containing as active agent ectoine, hydroxyectoine and/or salts, esters or amides of these compounds for use in a method for the treatment and/or prophylaxis of gastroesophageal reflux diseases, inflammation of and damage to the gastric or duodenal mucosa and/or of gastric or duodenal ulcers.

2. Composition for use according to claim 1, characterized in that the gastroesophageal reflux disease is a reflux esophagitis.

3. Composition for use according to claim 1, characterized in that the gastroesophageal reflux disease is a non-erosive reflux disease.

4. Composition for use according to claim 1, characterized in that the gastroesophageal reflux disease is a Barret's esophagus.

5. Composition for use according to claim 1, characterized in that the composition is an aqueous solution.

6. Composition for use according to claim 1, characterized in that the composition is a composition for oral administration.

7. Composition for use according to claim 1, characterized in that the concentration of ectoine, hydroxyectoine and/or salts, esters or amides of these compounds in the composition ranges between 10 and 500 mM.

8. Composition for use according to claim 7, characterized in that the concentration of ectoine, hydroxyectoine and/or salts, esters or amides of these compounds in the composition ranges between 100 and 500 mM.

9. Composition for use according to claim 1, characterized in that the composition is encapsulated in nanostructures or is provided in the form of liposomes.
